(19) United States
(12) Patent Application Publication
(10) Pub. No.: US 2009/0240165 A1
(43) Pub. Date: Sep. 24, 2009

(54) Apparatus for containing sampled liquid

(75) Inventors: Noboru Yoneya, Tokyo (JP); Takayuki Nakayama, Tokyo (JP); Kazunori Aoyagi, Tokyo (JP); Mitsuo Fukuda, Nishinomiya-shi (JP)

Correspondence Address: EDWARDS ANGELL PALMER & DODGE LLP, P.O. BOX 55874, BOSTON, MA, 02205, US

(73) Assignee: LIGHTNIX, INC., Nishinomiya-shi (JP)

(21) Appl. No.: 12/382,432

(22) Filed: Mar. 17, 2009

(30) Foreign Application Priority Data
Sep. 6, 2007 (JP) 2007-231196

(51) Int. Cl. A61B 5/151

(52) U.S. Cl. 600/583

(57) ABSTRACT

An apparatus for containing sampled liquid includes: a substrate 11; and a puncture portion 13, integral with the substrate 11, and that projects out of the substrate 11 for insertion into an organism. The puncture portion 13 includes a collection channel 17, and the substrate 11 includes a storage channel 15 in communication with the collection channel 17. The collection channel 17 collects a compositional bodily fluid, and the storage channel 15 stores the compositional bodily fluid transferred thereto by capillary action. The storage channel 15 includes an inflecting portion or a branched portion shaped to enable capillary action transfer of the compositional bodily fluid. At least an innermost portion 36 of the storage channel 15 farthest from the collection channel 17 is open to atmosphere.

### BACKGROUND OF THE INVENTION

(1) Field of the Invention

The present invention relates to an apparatus for containing sampled liquid punctured into an organism to collect a compositional bodily fluid, and that stores the collected compositional bodily fluid to enable a measurement, analysis, or other processing of the compositional bodily fluid.

(2) Description of the Related Art

Devices that collect a compositional bodily fluid to perform various tests are known in which a portion to collect a compositional bodily fluid from an organism is integrated into a portion where the measurement, analysis, or other processing of the collected compositional bodily fluid are performed.

Patent Document 1 proposes a blood collecting device in which a blood collection needle punctured into an organism to collect blood projects out of the main body integral with the needle. In this blood collecting device, the blood collection needle projects out of the periphery of the main body formed from, for example, an SOI plate. The blood collection needle includes a blood-collecting hollow portion in communication with a meandering channel provided in the main body. The device also includes a pump unit at an end of the channel. The blood collection needle is punctured into an organism, and the pump unit is used to draw blood into the channel, where the blood glucose level or other characteristics of the blood is measured. Because this type of blood collecting device requires a driving section, such as the pump unit, to draw the blood into the meandering channel, the configuration of the device tends to become complex.

Patent Document 2 proposes a device for analyte measurement configured so that a lance including a lancing element, a separation element, and a connector connecting these elements projects out of a substrate integral with the lance. In this device, a fill channel is provided that extends in a straight line from the gap between the lancing element and the separation element of the connector to the measurement site of the substrate. The tip of the lancing element is used to make an incision in the skin, and bodily fluid is collected in the gap between the lancing element and the separation element. The measurement of the bodily fluid is made by transferring the bodily fluid to the measurement site through the fill channel by capillary action. This device can transfer the bodily fluid to the measurement site without using driving means such as a pump unit. However, when the device is reduced in size, the fill channel becomes short, and it becomes difficult to store a sufficient amount of bodily fluid.

Patent Document 3 proposes a test strip device that includes a substrate and a microneedle. The microneedle is integral with the substrate, and two-dimensionally extends out of the substrate. An opening to pool bodily fluid is provided in the microneedle, and a channel in communication with the opening extends to the substrate. The channel has large numbers of sub-channels, above which a continuous open space is created to provide a reaction zone or the like. In this test strip device, the microneedle is punctured into an organism to pool bodily fluid in the opening of the microneedle. The bodily fluid is then stored in the channel by being drawn or by capillary action. The bodily fluid in the channel is stored in the sub-channels, and, when stored in sufficient amounts, fills the reaction zone or the like, where various measurements are performed. In this device, it is difficult to smoothly store a sufficient amount of bodily fluid in the reaction zone or the like by capillary action through the opening, channel, and the large numbers of sub-channels, and the storage of a sufficient amount of bodily fluid often takes time.
Patent Document 1: JP-A-2000-185034
Patent Document 2: JP-A-2004-298628
Patent Document 3: JP-A-2004-113772

### BRIEF SUMMARY OF THE INVENTION

In devices in which the puncture portion used to collect a compositional bodily fluid is integrated into a portion where the collected compositional bodily fluid undergoes a measurement, analysis, or some other processing, it is difficult to ensure a desired storage volume or storage speed because, when a sufficient amount of compositional bodily fluid is to be stored by capillary action without using a driving section such as a pump, a sufficient force cannot be provided for the transfer of the compositional bodily fluid, and the storage volume or storage speed tends to vary greatly depending on the channel configuration.

Ensuring a sufficient storage volume is particularly difficult when the size of the fine puncture portion is reduced to relieve the burden on an organism, because it accompanies a corresponding reduction in the size of the collecting portion or the channel.

It is accordingly an object of the present invention to provide an apparatus for containing sampled liquid that can store a sufficient amount of compositional bodily fluid in a short time period with a simple configuration.

The invention provides an apparatus for containing sampled liquid that includes: a substrate; and a puncture portion, integral with the substrate, and that projects out of the substrate for insertion into an organism. The puncture portion includes a collection channel, and the substrate includes a storage channel in communication with the collection channel. The collection channel collects a compositional bodily fluid, and the storage channel stores the compositional bodily fluid transferred thereto by capillary action. The storage channel includes an inflecting portion or a branched portion shaped to enable capillary action transfer of the compositional bodily fluid. At least an innermost portion of the storage channel farthest from the collection channel is open to atmosphere.

The provision of the inflecting portion or branched portion shaped to enable capillary action transfer of the compositional bodily fluid helps increase the total length of the storage channel, and therefore the total volume of the storage channel. Because the increased storage channel length helps increase the inner wall surface area in contact with the compositional bodily fluid in the storage channel for a given storage volume of the compositional bodily fluid, the capillary force required for the transfer of the compositional bodily fluid can be provided with ease. This facilitates the storage of a sufficient amount of compositional bodily fluid in the storage channel in a short time period. Further, because the compositional bodily fluid is stored in the storage channel by capillary action transfer, the device does not require driving means to collect the compositional bodily fluid, making it possible to simplify the configuration.

### ADVANTAGEOUS EFFECTS

The invention provides an apparatus for containing sampled liquid that includes a storage channel provided with an inflecting portion or a branched portion shaped to enable capillary action transfer of a compositional bodily fluid. In the storage channel, at least an innermost portion farthest from the collection channel is open to atmosphere. With this construction, the apparatus for containing sampled liquid can easily store a sufficient amount of compositional bodily fluid in a short time period with a simple configuration.

### BRIEF DESCRIPTION OF SEVERAL VIEWS OF THE DRAWING

FIG. 1 is a perspective view illustrating an apparatus for containing sampled liquid of an embodiment of the present invention.

FIG. 2A through FIG. 2C illustrate a puncture portion of the apparatus for containing sampled liquid of an embodiment of the present invention, in which FIG. 2A is a plan view, FIG. 2B is a side view, and FIG. 2C is a front view.

FIG. 3A through FIG. 3C are partial enlarged views of storage channels of the apparatus for containing sampled liquid of an embodiment of the present invention, in which FIG. 3A is a plan view, FIG. 3B is a cross sectional view taken at line A-A of FIG. 3A, and FIG. 3C is a cross sectional view taken at line B-B of FIG. 3A.

FIG. 4 is a plan view of a substrate according to a modification example of the apparatus for containing sampled liquid of an embodiment of the present invention.

FIG. 5A through FIG. 5G are photographs showing test pieces tested in Examples 1 to 7.

### DETAILED DESCRIPTION OF THE INVENTION

An embodiment of the present invention is described below with reference to the accompanying drawings.

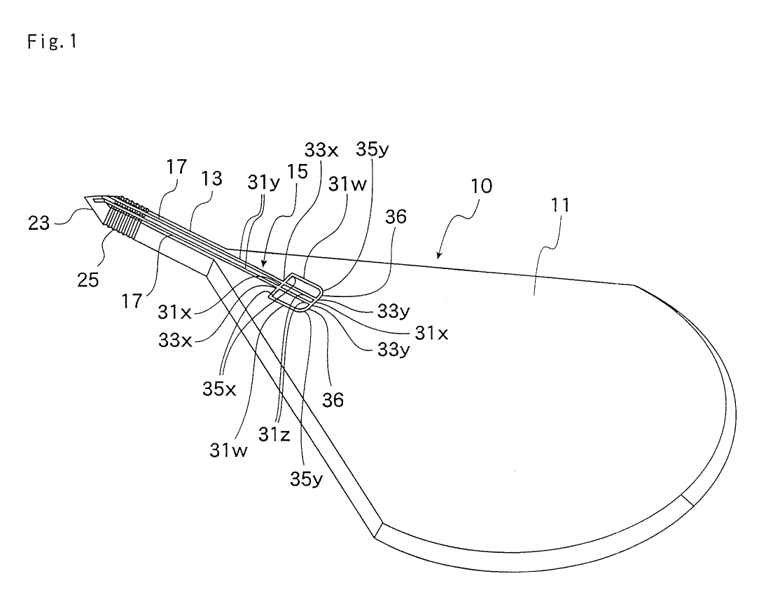

FIG. 1 through FIG. 3 represent an embodiment of the present invention.

FIG. 1 illustrates an apparatus for containing sampled liquid 10. The apparatus for containing sampled liquid 10 includes a plate-like substrate 11, and a puncture portion 13 integral with the substrate 11, and that projects out as a partial protrusion from the periphery of the substrate 11 in the plane of the plate-like substrate 11. The puncture portion 13 includes collection channels 17 formed from micro channels. The substrate 11 includes storage channels 15 formed from micro channels in communication with the collection channels 17.

The apparatus for containing sampled liquid 10 is a device that enables a measurement, analysis, or other processing of a compositional bodily fluid. This is attained by puncturing the puncture portion 13 into an organism, collecting the compositional bodily fluid in the collection channels 17, and storing the compositional bodily fluid in the storage channels 15 by the capillary action transfer of the fluid to the storage channels 15.

The compositional bodily fluid to be collected by the apparatus for containing sampled liquid 10 is a variety of fluids found in organisms, such as the blood and the interstitial fluid of humans and animals. The processing of the compositional bodily fluid includes various processes performed on small quantities of compositional bodily fluid, for example, such as a measurement or analysis of various components contained in the compositional bodily fluid, and a reaction of the compositional bodily fluid with other components. Examples include measurements of blood glucose level; blood tests for ketone bodies, glycohemoglobin, lipids, proteins, and antigen-antibody reaction; DNA analysis; identification of antibodies and proteins, and testing of chemical substances.

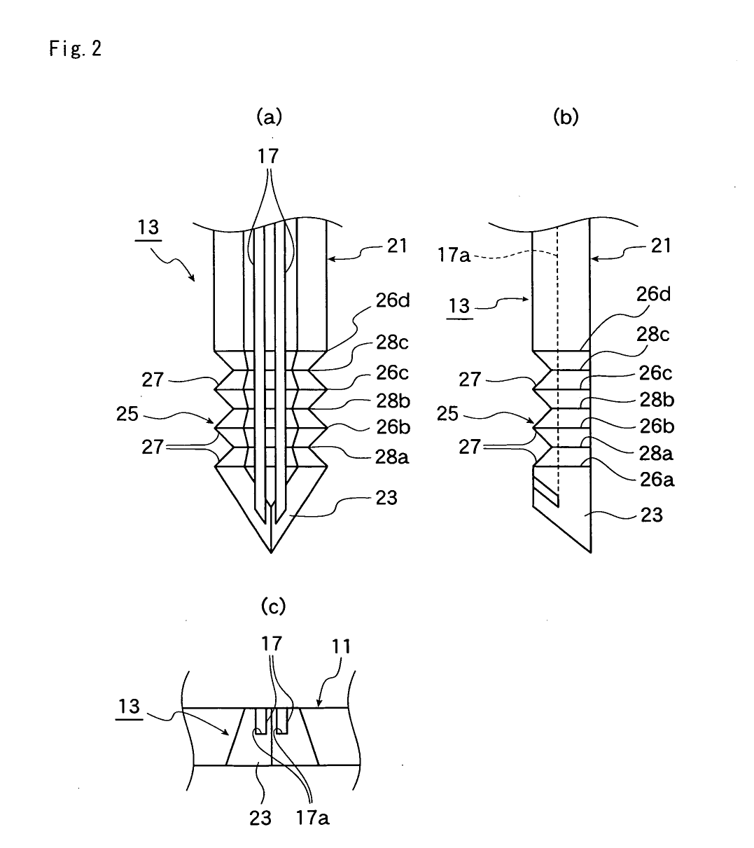

The puncture portion 13 of the apparatus for containing sampled liquid 10 is described below. In this embodiment, as illustrated in FIG. 2, the puncture portion 13 includes a base end 21 on the substrate 11 side, an end point 23 at the tip, and an jagged surface portion 25 having jags formed on the lateral periphery. The cross section of the base end 21, the end point 23, and the jagged surface portion 25 orthogonal to the projecting direction of the puncture portion 13 is trapezoidal in shape over the entire length.

The size of the puncture portion 13 can be appropriately selected according to intended use. For example, the maximum length of the cross section orthogonal to the direction of projection from the substrate 11 may fall in a range of from 100 to 2,000 μm, and the total length along the projecting direction may be from 0.15 to 2 mm. When the maximum length of the cross section orthogonal to the projecting direction is excessively small, it becomes difficult to ensure sufficient strength during use. Further, it makes the collection channels 17 too narrow to collect a sufficient amount of fluid. On the other hand, when the maximum length of the cross section is too large, the puncturing causes more damage in the surrounding cells or more pain in the organism, increasing the burden on the organism. When the total length along the projecting direction is too small, it becomes difficult to puncture the organism. When too large, it becomes difficult to provide strength for the puncture portion 13 during use, and bending or breaking becomes likely during puncturing.

The cross sectional shape of the puncture portion 13 orthogonal to the projecting direction can be suitably selected. For example, the cross section may be semicircular, circular, semi-elliptical, elliptical, triangular, square, rectangular, trapezoidal, rhomboidal, polygonal (including a pentagon and higher polygons), and various jagged shapes. In this embodiment, as illustrated in FIG. 2, the cross section is trapezoidal in shape for ease of formation and other considerations.

The jagged surface portion 25 is provided on preferably at least a portion of the lateral periphery of the puncture portion 13. Particularly preferably, ridge portions 27 are repeatedly formed along the projecting direction of the puncture portion 13. In this case, it is particularly preferable that maximum points 26a, 26b, and 26c, at which the cross sectional area orthogonal to the projecting direction of the puncture portion 13 is maximum, be alternately disposed with minimum points 28a, 28b, and 28c, at which the cross sectional area is minimum, and that the cross sectional area at the maximum point 26a closest to the tip be equal to or greater than the cross sectional area at the maximum points 26b and 26c on the side of the substrate 11. This helps reduce damage to the surrounding cells or pain caused by puncturing, reducing the burden on the organism. The shape and the position of the jagged surface portion 25 can be suitably selected according to such factors as the intended use of the apparatus for containing sampled liquid 10, and the site of puncturing in an organism.

For purposes such as the reduction of the burden on an organism, and the provision of strength for the puncture portion 13 during use, it is preferable that the maximum length of the cross section at the maximum points 26a, 26b, and 26c be 40 to 600 μm, more preferably 80 to 300 μm, and that the maximum length of the cross section at the minimum points 28a, 28b, and 28c be 20 to 300 μm, more preferably 40 to 150 μm, and that the distance between the maximum points 26a, 26b, and 26c be 5 to 200 μm, more preferably 20 to 100 μm.

The collection channels 17 of the puncture portion 13 are configured to collect the compositional bodily fluid of an organism upon insertion, and transfer the fluid to the storage channels 15 of the substrate 11 by capillary action. The collection channels 17 may be provided as hollow pores formed inside the puncture portion 13, opening to outside at a predetermined position on the tip side and/or the lateral periphery of the puncture portion 13. Alternatively, the collection channels 17 may be provided as grooves or slits opening to outside on the lateral periphery of the puncture portion 13. Preferably, the collection channels 17 are provided as grooves, because the grooves are easy to form, and help increase the contact area with the compositional bodily fluid, and provide sufficient strength for the puncture portion 13 during use.

When the collection channels 17 are provided as hollow pores or grooves, the cross section of the collection channels 17 orthogonal to the projecting direction of the puncture portion 13 can have any shape, for example, such as an arc, a substantially triangular shape (substantially V-shaped), and a substantially quadrangular shape (substantially U-shaped). The cross section preferably has the same shape as that of the storage channels 15 of the substrate 11, from the standpoint of preventing formation of steps between the collection channels 17 and the storage channels 15, as will be described later.

In this embodiment, the collection channels 17 are provided as two grooves along the projecting direction of the puncture portion 13, and the cross section of each collection channel 17 orthogonal to the projecting direction of the puncture portion 13 is substantially quadrangular in shape (substantially U-shaped). The cross section is so shaped because it helps increase the cross sectional area for a given opening width. Each collection channel 17 is continuously provided on the jagged surface portion 25, passing through the ridge portions 27 in the projecting direction of the puncture portion 13. The inner wall surfaces of each collection channel 17 include a flat bottom surface 17a extending along the entire length.

Preferably, the cross sectional area of the collection channels 17 orthogonal to the projecting direction of the puncture portion 13 is set so that a sufficient strength is provided for the puncture portion 13 during use, and that the collected compositional bodily fluid can be transferred at a sufficient transfer speed by capillary action.

For example, when the storage volume of compositional bodily fluid in the storage channels 15 of the substrate 11 is 10 to 600 nl, the cross sectional area may be 200 μm² or more, or may be the same as the cross sectional area of the storage channels 15 of the substrate 11 (described later), for purposes such as the collection of a sufficient amount of fluid.

Further, when the collection channels 17 are provided as grooves, the opening width may be the same as that of the storage channels 15 of the substrate 11. For example, when the storage volume of compositional bodily fluid in the storage channels 15 of the substrate 11 is 10 to 600 nl, the opening width may be 10 to 30 μm.

When the collection channels 17 are provided as grooves on the puncture portion 13 having the jagged surface portion 25 on the lateral periphery, it is preferable that the collection channels 17 continuously open to outside on the surface of the jags. In this way, the collection channels 17 can have a wide contact area with the compositional bodily fluid, facilitating the fluid collection.

The collection channels 17 may be provided as a single channel. However, providing a plurality of collection channels 17 as in this embodiment is more preferable because it helps increase the amount of collection, and the area of the inner wall surfaces in contact with the compositional bodily fluid.

The substrate 11 of the apparatus for containing sampled liquid 10 is described below.

As illustrated in FIG. 1, the substrate 11 is planar in shape, and includes the puncture portion 13 in such an arrangement that the puncture portion 13 is puncturable into an organism. The storage channels 15 in communication with the collection channels 17 are provided on one side of the substrate 11. For handling such as operability, the portion of the substrate 11 opposite from the puncture portion 13 is provided with a receptacle where the storage channels 15 are not disposed.

The shape, size, or other configuration of the substrate 11 is not particularly limited, as long as the puncture portion 13 can be provided, and the storage channels 15 can be formed to ensure a desired storage volume.

The storage channels 15 of the substrate 11 are configured so that the compositional bodily fluid transferred from the collection channels 17 of the puncture portion 13 can be stored therein by capillary action transfer. The storage channels 15 are compartmentalized by their inner wall surfaces along the axis line. It is not preferable to slope the inner wall surfaces with an abrupt angle with respect to the axis line of the storage channels 15 over the entire length.

The storage channels 15 may be open to atmosphere over the entire length, or may be hollow pores except at an innermost portion 36 open to atmosphere. With the storage channels 15 open to atmosphere, the gas inside the storage channels 15 can easily be released during the transfer and storage of the compositional bodily fluid. With the storage channels 15 provided as hollow pores, the contact area with the compositional bodily fluid transferred in the storage channels 15 can be increased to help increase the capillary force for the transfer of the compositional bodily fluid. The hollow pores can be formed by covering the grooved storage channels with a member such as a plate. For example, a plate prepared to include grooves symmetrical to the storage channels 15 is mated with the storage channels 15 to form the hollow pores.

In this embodiment, the storage channels 15 are provided as grooves open to atmosphere over the entire length. The grooved channels are easy to form, and enable the gas inside the storage channels 15 to be easily released. The open channel configuration is preferable because it helps release the gas from the channels and enables the transfer and storage of the compositional bodily fluid introduced from different directions at the same time, particularly in partial storage channels 31x configured to introduce the compositional bodily fluid from different directions, as will be described later.

As illustrated in FIG. 1, the storage channels 15 are configured to include branched portions 33x and 33y, and bent or curved inflecting portions 35x and 35y, spreading out two-dimensionally in the plane of the substrate 11. Each branched portion 33x is in communication with the partial storage channel 31x disposed between the branched portions 33x; partial storage channels 31y in communication with the collection channels 17; partial storage channels 31z linearly disposed between the branched portions 33x and the branched portions 33y; and partial storage channels 31w disposed with inflecting portions 35x and 35y between the branched portions 33x and the branched portions 33y. Each branched portion 33y is in communication with the partial storage channel 31x disposed between the branched portions 33y; the partial storage channels 31z; and the partial storage channels 31w. In the storage channels 15, the innermost portion 36 is defined by the partial storage channels 31w.

By the provision of the branched portions 33x and 33y, the compositional bodily fluid can be transferred by being branched into the partial storage channels 31w, 31x, and 31z. This configuration is more preferable than the arrangement without the branched portions 33x and 33y, because it reduces the transfer distance of the compositional bodily fluid in the storage channels 15, and therefore the storage time of the compositional bodily fluid.

The cross section of the partial storage channels 31w, 31x, 31y, 31z of the storage channels 15 orthogonal to the axis line may have any shape, such as an arc, a substantially triangular shape (substantially V-shaped), or a substantially quadrangular shape (substantially U-shaped). It is preferable that the partial storage channels 31w, 31x, 31y, 31z in communication with one another via the branched portions 33x and 33y have the same or similar cross sectional shape orthogonal to the axis line. With the same or similar cross sectional shape, the cross sections of these portions will resemble or match along the entire channels. This helps prevent formation of steps between the inner wall surfaces of the partial storage channel 31w, 31x, 31y, 31z at the branched portions 33x and 33y, particularly when the channels have the same cross sectional shape. Here, the partial storage channel 31w, 31x, 31y, 31z are all substantially quadrangular in shape (substantially U-shaped), because helps increase the cross sectional area for a given opening width.

The storage channels 15 must have a volume large enough to store a sufficient amount of compositional bodily fluid for intended measurements, analyses, or other processing. For example, the total storage volume of the storage channels 15 for the compositional bodily fluid may be 10 to 600 nl.

It is preferable that the cross sectional areas of the partial storage channels 31w, 31x, 31y, 31z orthogonal to the axis line be set to enable a desired amount of compositional bodily fluid to be transferred by capillary action at a sufficient transfer speed. For example, when the storage volume of compositional bodily fluid in the storage channels 15 is 10 to 600 nl, the cross sectional area is preferably 200 to 1,800 μm². When the cross sectional area is excessively small, the entire length of the partial storage channels 31w, 31x, 31y, 31z tends to increase, which increases the space required to dispose the storage channels 15. Further, an excessively small cross sectional area causes undesirable effects such as increased fluid resistance, and the speed of capillary action transfer tends to decrease. On the other hand, when the cross sectional area is too large, the surface tension for a given storage volume of the compositional bodily fluid tends to decrease, which tends to reduce the speed of capillary action transfer. The cross sectional area may be continuously increased or decreased along the axis line within the same partial storage channels 31w, 31x, 31y, and 31z.

The opening width of the grooved, partial storage channels 31w, 31x, 31y, 31z may be set so as to make molding of uniform inner wall surfaces easier, and to obtain a sufficient surface tension. For example, when the storage volume of the storage channels 15 is 10 to 600 nl, the opening width may be 10 to 30 μm.

It is preferable that the inner wall surfaces of the partial storage channels 31y in communication with the collection channels 17 of the puncture portion 13 be continuous to the inner wall surfaces of the collection channels 17 without any steps. This helps the capillary action transfer of the compositional bodily fluid from the collection channels 17 to the storage channels 15. Here, the collection channels 17 of the puncture portion 13, and the partial storage channels 31y have the same cross sectional shape orthogonal to the axis line, and are continuous without any clear boundary.

In the storage channels 15, when the inner wall surfaces have an altered shape portion where the shape of the storage channels 15 changes, the contact state between the compositional bodily fluid and the inner wall surfaces, for example, such as the wettability of the inner wall surfaces, and fluid resistance, tend to change in portions separated by the altered shape portion. This makes it difficult to smoothly transfer the compositional bodily fluid, particularly when there is an abrupt change in the altered shape portion, such as in large steps, and discontinuous portions on the inner wall surfaces.

Because the storage channels 15 includes the inflecting portions 35x and 35y, at least a part of the inner wall surfaces at these portions undergoes a geometrical change by being curved or bent. Further, at the branched portions 33x and 33y, because at least three partial storage channels, 31w, 31x, 31y, or 31z, are in communication with one another, at least a part of the inner wall surfaces undergoes a geometrical change by being curved or bent, while the other inner wall surfaces are discontinuous. Thus, the inflecting portions 35x and 35y and the branched portions 33x and 33y tend to prevent a smooth capillary action transfer of the compositional bodily fluid, and storage of a sufficient amount of compositional bodily fluid in a short time period. The transfer of the compositional bodily fluid may not be possible at all when there is a step or other obstacles in these portions.

To avoid such problems, the inflecting portions 35x and 35y and the branched portions 33x and 33y in the storage channels 15 must be shaped to enable capillary action transfer of the compositional bodily fluid.

In one exemplary configuration of the inflecting portions 35x and 35y and the branched portions 33x and 33y that enables capillary action transfer of the compositional bodily fluid, it is preferable that at least a part of the inner wall surfaces of the storage channels 15 be continuous in the inflecting portions 35x and 35y and the branched portions 33x and 33y without substantially any step. As used herein, “to be continuous without substantially any step” means that the surfaces are continuous without any portions where the wettability of the compositional bodily fluid transferred in the storage channels 15 varies discontinuously, or portions where the fluid resistance for the transferred compositional bodily fluid varies discontinuously, such as in projections or other jags, and bent surfaces with an apex.

For example, at the inflecting portions 35x and 35y, at least a part of the inner wall surfaces, preferably all of the inner wall surfaces may be flat or curved surfaces that are smoothly continuous to the adjacent inner wall surfaces of the storage channels 15 on the both sides. Further, at the branched portions 33x and 33y, when the cross sectional shape of the partial storage channels 31w, 31x, 31y, and 31z is substantially quadrangular (substantially U-shaped), at least a part of the inner wall surfaces, preferably a pair of the inner wall surfaces in the plane of the substrate 11 may be flat or curved surfaces that are smoothly continuous to the inner wall surfaces of the partial storage channels 31w, 31x, 31y, and 31z in communication with one another via the branched portions 33x and 33y. Further, when the cross sectional shape of the partial storage channels 31w, 31x, 31y, and 31z is substantially triangular (substantially V-shaped) or arc-like, the inner wall surface(s) and/or the edge facing the inner wall surface in the plane of the substrate 11 may be a flat or curved surface(s), or a straight or curved line, that is smoothly continuous to the inner wall surfaces and/or the edges of the partial storage channels 31w, 31x; 31y, and 31z in communication with one another via the branched portions 33x and 33y.

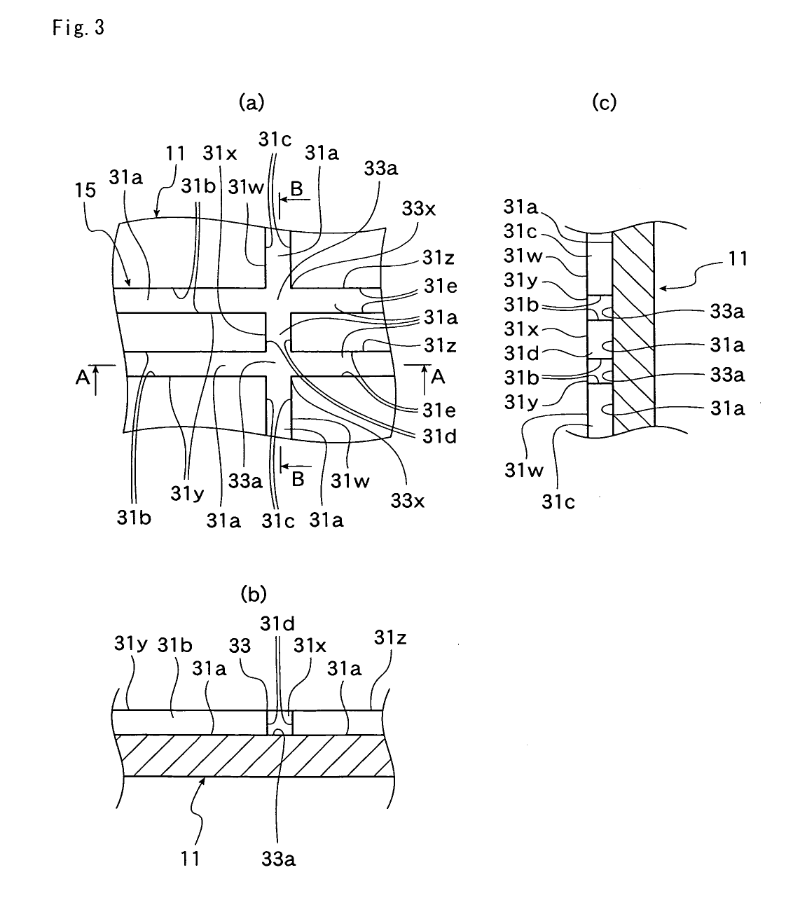

In this embodiment, the cross sectional shapes of the partial storage channels 31w, 31x, 31y, and 31z are substantially quadrangular (substantially U-shaped). At the inflecting portions 35x and 35y, the bottom surface of the groove in the partial storage channels 31w is a smooth, continuous flat surface, and the side surfaces of the groove are smooth, continuous curved surfaces. At the branched portions 33x and 33y, as illustrated in FIG. 3A through FIG. 3C, bottom surfaces 33a of the groove are flat surfaces that are smoothly continuous to bottom surfaces 31a of the partial storage channels 31. Further, the bottom surfaces 31a of the groove in the partial storage channels 31w, 31x, 31y, and 31z, and the bottom surfaces 33a in the branched portions 33x and 33y form a flat, continuous surface over the entire length of the storage channels 15 within the two-dimensional plane of the substrate 11. The depth of the groove is uniform. This makes it easier to transfer the transferred compositional bodily fluid along the bottom surfaces 31a and 33a, facilitating a smooth transfer of the compositional bodily fluid throughout the storage channels 15.

In another exemplary configuration of the inflecting portions 35x and 35y and the branched portions 33x and 33y that enables capillary action transfer of the compositional bodily fluid, the partial storage channels 31w, 31x, 31y, and 31z may be formed so that a maximum value-to-minimum value ratio of the channel cross sectional area orthogonal to the axis line of these channels is 2.5 or less at the ends of the partial storage channels 31w, 31x, 31y, and 31z adjacent to the branched portions 33x and 33y. In this range, there will not be large differences in the capillary force transferring the compositional bodily fluid, or in the fluid resistance between the partial storage channels 31w, 31x, 31y, and 31z, making it possible to equally transfer the compositional bodily fluid to the partial storage channels 31w, 31x, 31y, and 31z. Here, the maximum value-to-minimum value ratio of the cross sectional area orthogonal to the axis line of the channels is preferably 2.5 or less throughout the storage channels 15 except at the branched portions 33x and 33y. It is particularly preferable, as in this embodiment, that the cross sections of all the partial storage channels 31w, 31x, 31y, and 31z orthogonal to the axis line have the same shape over the entire length, because it helps prevent formation of large steps at the branched portions 33x and 33y.

As required, electrodes or various other components, or chemicals such as enzymes and DNA fragments for the measurement, analysis, or other processing of the compositional bodily fluid may be disposed on the substrate 11, in contact with the storage channels 15.

The apparatus for containing sampled liquid 10 is made of biocompatible material. Examples of the biocompatible material include high molecular polymers, biopolymers, proteins, and biocompatible inorganic materials.

The high molecular polymer is preferably those usable in medical applications, for example, such as polyvinyl chloride, polyethylene glycol, parylene, polyethylene, polypropylene, silicone, polyisoprene, polymethyl methacrylate, fluorocarbon resin, polyether imide, polyethylene oxide, polyethylene terephthalate, polyethylene succinate, polybutylene terephthalate, polybutylene succinate, polybutylene succinate carbonate, polyphenylene oxide, polyphenylene sulfide, polyformaldehyde, polyanhydride, polyamide (nylon 6, nylon 66), polybutadiene, polyvinyl acetate, polyvinyl alcohol, polyvinyl pyrrolidone, polyester amide, polymethyl methacrylate, polyacrylonitrile, polysulfone, polyethersulfone, ABS resin, polycarbonate, polyurethane (polyether urethane, polyester urethane, polyether urethane urea), polyvinylidene chloride, polystyrene, polyacetal, polybutadiene, ethylene-vinyl acetate copolymer, ethylene-vinyl alcohol copolymer, ethylene-propylene copolymer, polyhydroxyethyl methacrylate, polyhydroxybutyrate, poly(ortho ester), polylactic acid, polyglycol, polycaprolactone, polylactic acid copolymer, polyglycolic acid.glycol copolymer, polycaprolactone copolymer, polydioxanone, perfluoroethylene-propylene copolymer, cyanoacrylate polymer, polybutyl cyanoacrylate, polyallyl ether ketone, epoxy resin, polyester resin, polyimide, phenol resin, and acrylic resin.

Examples of the biopolymer include cellulose, starch, chitin.chitosan, agar, carrageenan, alginic acid, agarose, pullulan, mannan, curdlan, xanthan gum, gellan gum, pectin, xyloglucan, guar gum, lignin, oligosaccharide, hyaluronan, schizophyllan, and lentinan. Examples of the protein include collagen, gelatin, keratin, fibroin, glue, sericin, vegetable protein, milk protein, egg protein, synthetic protein, heparin, and nucleic acid. Other examples include sugars, syrup, glucose, maltose, sucrose, and a polymer alloy thereof.

Examples of the biocompatible inorganic material include: ceramics such as glass; nanocomposite ceramics; Al₂O₃/ZrO₂ composite ceramics; Si₃N₄ nanocomposite materials; hydroxyapatite; calcium carbonate; carbon; graphite (nanografiber); carbon nanotubes (CNT); fullerene composite materials; hydroxyapatite.polymer composite materials; cobalt-chromium alloys; stainless steel; titanium; and titanium alloys.

Among these biocompatible materials, it is preferable to use biodegradable polymers that contain materials, for example, such polylactic acid, polyglycolic acid, polycaprolactone, collagen, starch, hyaluronan, alginic acid, chitin, chitosan, cellulose, and gelatin. Use of biodegradable materials made of these compounds is also preferable. These materials are preferable because they decompose in a microbe environment, which makes the disposal of the apparatus for containing sampled liquid 10 easier after use.

Use of polylactic acid is particularly preferable. Because polylactic acid is moderately compatible to the compositional bodily fluid, it makes it easier to collect, transfer, and store the compositional bodily fluid by capillary action through the collection channels 17 and the storage channels 15. Polylactic acid also helps prevent excessive adsorption of the components contained in the compositional bodily fluid.

In the apparatus for containing sampled liquid 10 made of biocompatible materials, it is preferable to perform a biocompatibility enhancing treatment on the surface of the apparatus for containing sampled liquid 10, or at least on the inner wall surfaces of the collection channels 17 and the storage channels 15.

The biocompatibility enhancing treatment is intended to adjust compatibility to the compositional bodily fluid, or help prevent adsorption of the compositional bodily fluid components, by modifying the surface in contact with the compositional bodily fluid, or applying a surface treatment agent.

The biocompatibility enhancing treatment that adjusts compatibility to the compositional bodily fluid can be performed, for example, by applying and immobilizing chemicals such as polyethylene glycol, sodium hydroxide, polysorbate, Poloxamer, and silicone.

The biocompatibility enhancing treatment that helps prevent adsorption of the compositional bodily fluid components can be performed, for example, by applying and immobilizing chemicals such as heparin, phosphoric acid, polyethylene glycol, sodium hydroxide, polysorbate, Poloxamer, and silicone.

The compatibility to the compositional bodily fluid can be evaluated by, for example, the size of the contact angle, although the invention is not so limited. With the compatibility appropriately selected this way, the capillary action collection and storage of the compositional bodily fluid in the collection channels 17 and the storage channels 15 becomes easier. It is preferable to prevent adsorption of the compositional bodily fluid components as much as possible, and, for this purpose, the agent may be appropriately applied to such an extent that the compatibility to the compositional bodily fluid is not inhibited.

The method for producing the apparatus for containing sampled liquid 10 configured as above is not particularly limited. In one exemplary method, the puncture portion 13 and the substrate 11 are molded as an integral unit using the foregoing materials, and the collection channels 17 and the storage channels 15 are formed thereon using, for example, an excimer laser. In another exemplary method, the puncture portion 13 and the substrate 11 are formed as an integral unit using a mold having patterns for the collection channels 17 and the storage channels 15, followed by various biocompatibility enhancing treatments as required.

The measurement, analysis, or other processing of the compositional bodily fluid using the apparatus for containing sampled liquid 10 can be performed as follows.

First, the apparatus for containing sampled liquid 10 illustrated in FIG. 1 is held on an operating holder (not shown) using the receptacle of the substrate 11 on the opposite end of the puncture portion 13. By holding the operating holder, the puncture portion 13 is punctured into an organism from the side of the end point 23. The insertion of the puncture portion 13 places the jagged surface portion 25 inside the organism, and the openings of the collection channels 17 continuously formed along the jags of the jagged surface portion 25 will be in contact with the compositional bodily fluid. The compositional bodily fluid in contact with the openings of the collection channels 17 is then collected into the collection channels 17 under the pressure of the organism and by capillary action.

The compositional bodily fluid in each collection channel 17 is transferred toward the substrate 11 by capillary action as it is collected. The capillary action transfers the compositional bodily fluid from the collection channels 17 to the partial storage channels 31y in communication with the collection channels 17. Here, because the inner wall surfaces of the collection channels 17 are continuous to the inner wall surfaces of the partial storage channels 31 without steps, the compositional bodily fluid in the collection channels 17 is smoothly transferred into the partial storage channels 31y. The transferred compositional bodily fluid reaches the branched portions 33x.

At each branched portion 33x, as illustrated in FIG. 3A through FIG. 3C, side surfaces 31b of each partial storage channel 31y bend and join to one of the side surfaces 31c of the partial storage channel 31w, and one of the side surfaces 31d of the partial storage channel 31x. The side surfaces 31b are discontinuous to the other surfaces of the side surfaces 31c and 31d. The side surfaces 31b of each partial storage channel 31y are also discontinuous to side surfaces 31e of the partial storage channel 31z. However, because the bottom surface 31a of the partial storage channels 31y is a flat surface that is continuous to the bottom surfaces 31a of the other partial storage channels 31w, 31x, and 31z via the bottom surface 33a of each branched portion 33x, the compositional bodily fluid that has reached the branched portions 33x is equally introduced, via the bottom surface 33a of each branched portion 33x, to the bottom surfaces 31a of the partial storage channels 31w, 31x, and 31z connected to the branched portions 33x.

The compositional bodily fluid introduced into the partial storage channels 31w, 31x, and 31z is further transferred by capillary action in each channel. The compositional bodily fluid transferred through the partial storage channels 31z reaches the branched portions 33y, and, as in the branched portions 33x, is introduced into the partial storage channels 31x and 31w before it reaches the innermost portion 36 defined by the partial storage channel 31w. Here, the compositional bodily fluid is introduced in two different directions in the partial storage channel 31x connecting the branched portions 33x, and in each partial storage channel 31w connecting the branched portion 33x and the branched portion 33y. However, because the partial storage channels 31x and 31w are open to atmosphere, the gas inside the partial storage channels 31x and 31w can be released to outside, and the transfer and storage of the compositional bodily fluid is possible over the entire length of the partial storage channels 31x and 31w, even when the compositional bodily fluid is transferred from the both sides at the same time.

The storage of the compositional bodily fluid completes when the compositional bodily fluid is transferred throughout the storage channels 15.

The measurement, analysis, or other processing of the compositional bodily fluid stored in the storage channels 15 is performed in this state. This completes the use of the apparatus for containing sampled liquid 10.

In the apparatus for containing sampled liquid 10 as above, the compositional bodily fluid is transferred by capillary action and stored in the storage channels 15. Because this does not require driving means for the collection of the compositional bodily fluid, the apparatus for containing sampled liquid 10 can be realized with a simple configuration.

Further, because the storage channels 15 include the branched portions 33x and 33y and the inflecting portions 35x and 35y shaped to enable capillary action transfer of the compositional bodily fluid, the total length of the storage channels 15 can be increased by simply increasing the number of the partial storage channels 31w, 31x, 31y, and 31z. This helps increase the total volume of the storage channels 15. Further, the inner wall surfaces of the storage channels 15 in contact with the compositional bodily fluid can be increased for a given storage volume of the compositional bodily fluid. This helps increase the capillary force for the transfer of the compositional bodily fluid.

The storage channels 15 include a bent portion or a discontinuous portion in part of the inner wall surfaces at the branched portions 33x and 33y. However, because the inner wall surfaces of the storage channels 15 include a continuous bottom surface over substantially the entire length of the storage channels 15 without substantially any step, the capillary force for the transfer of the compositional bodily fluid from the partial storage channels 31y to the other partial storage channels 31w, 31x, and 31z via the branched portions 33x and 33y can easily be sustained to enable a smooth capillary action transfer of the compositional bodily fluid to the partial storage channels 31w, 31x, and 31z.

This facilitates the storage of a sufficient amount of compositional bodily fluid in the storage channels 15 in a short time period, and the measurement, analysis, and other processing of the compositional bodily fluid can be sufficiently performed with ease even when the number or size of the collection channels in the puncture portion 13 is restricted by the size of the puncture portion 13 reduced in size to relieve the burden on the organism undergoing the collection.

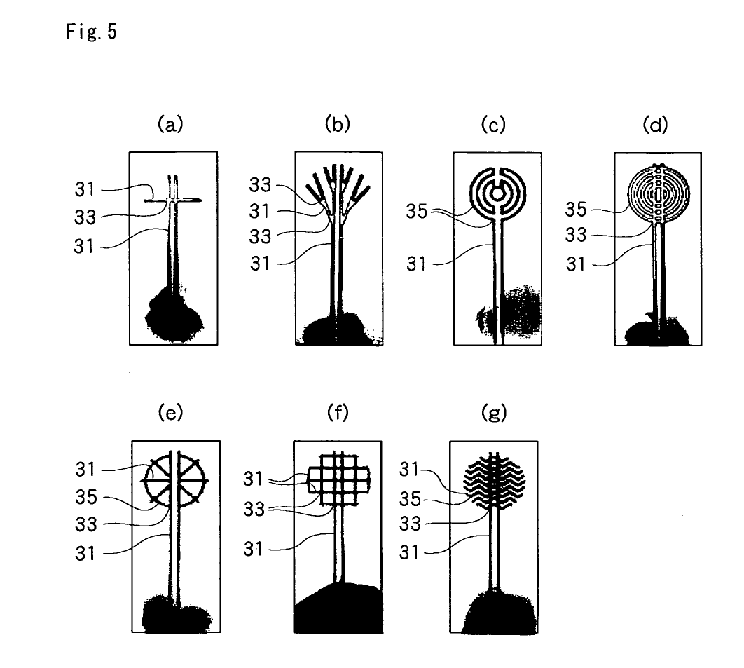

In this embodiment, the branched portions 33x and 33y are described as portions where the inner wall surfaces of the partial storage channels 31w, 31x, 31y, and 31z directly join. Alternatively, as illustrated in FIG. 5B (described later in conjunction with Examples), the inner wall surfaces of the partial storage channels 31 may be connected to each other via the inner wall surfaces of the branched portion 33, without an inflecting portion.

Further, the invention is not limited to the example in which the storage channels 15 include the branched portions 33x and 33y and the inflecting portions 35x and 35y. The configuration of the storage channels 15 can be appropriately designed. For example, as illustrated in FIG. 5C (described later in conjunction with Examples), channels with a plurality of inflecting portions may be provided, using only a few branched portions, if any.

In the exemplary configuration described above, the bottom surface 31a and the side surfaces of the partial storage channels 31y form flat, continuous surfaces with the bottom surface 17a and the side surfaces of the collection channels 17. The bottom surfaces 31a of the partial storage channels 31w, 31x, and 31z also form flat, continuous surfaces with the bottom surfaces 33a of the branched portions 33x and 33y. However, these continuous surfaces may be curved or slanted surfaces.

In this embodiment, the partial storage channels 31w, 31x, 31y, and 31z are described as having the same cross sectional shape orthogonal to the axis line. However, these cross sectional shapes may be different either entirely or partially. In this case, the partial storage channels 31w, 31x, 31y, and 31z may have different widths and the same depth, or may be shaped to have different depths.

Further, the invention is not limited to the arrangement in which the storage channels 15 in communication with the two collection channels 17 are one system of channels that are in communication with one another. The storage channels 15 in communication with the collection channels 17 may be channels of different systems that are not in communication with one another.

The storage channels 15 are described as being provided on one side of the substrate 11. However, the storage channels 15 may be provided on the both sides of the substrate 11 to help increase the storage volume.

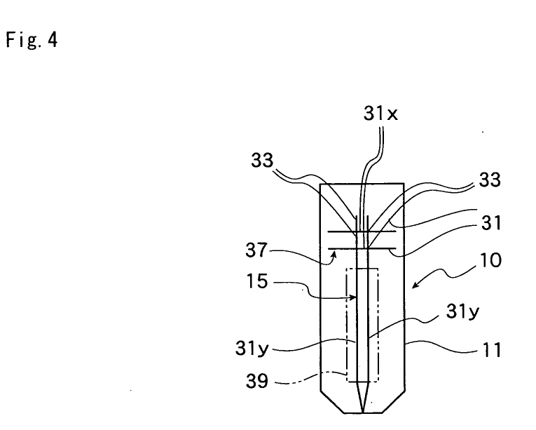

In the exemplary configuration above, the measurement, analysis, or other processing of the compositional bodily fluid may be performed in any portion in contact with the storage channels 15. However, the site of measurement, analysis, or other processing may be specifically designated. For example, as illustrated in FIG. 4, the partial storage channels 31 may be provided as large numbers of dense channels to form a dense portion 37 at a position distant from the puncture portion 13 of the substrate 11, and a measurement unit 39 may be provided in contact with the partial storage channels 31y disposed in communication with the collection channels 17 of the puncture portion 13 and the dense portion 37 in between. With this arrangement, the compositional bodily fluid collected in the collection channels 17 during the collection procedure can be transferred through the partial storage channels 31y by the capillary action driven by the dense portion 37, enabling the measurement, analysis, or other processing of the compositional bodily fluid. The dense portion 37 can thus be used as the driving section for the transfer of the compositional bodily fluid.

In this embodiment, the puncture portion 13 is described as having the jagged surface portion 25. However, the puncture portion 13 may have a uniform surface with no jags.

### EXAMPLES

Examples of the invention are described below.

### Examples 1 to 7

Seven kinds of test pieces were produced by injection molding using polylactic acid. Each test piece is a planar strip (length, 4 mm; width, 2 mm; thickness, 100 μm), and has planar storage channels 15, illustrated in FIG. 5A through FIG. 5G, and two collection channels 17 on one of the surfaces. The storage channels 15 and the collection channels 17 were formed as open grooves with substantially quadrangular (substantially U-shaped) cross sections (width, 20 μm; depth, 40 μm) orthogonal to the axis line.

Pseudo blood was dropped over the two collection channels 17 of each test piece, and the state of the pseudo blood was observed as it was transferred and stored in the storage channels 15.

The planar shape of the storage channels 15 of each test piece is as follows.

### Example 1

As illustrated in FIG. 5A, straight partial storage channels 31 are crossed at a right angle to form two branched portions 33. Each branched portion 33 is in communication with the straight partial storage channels 31 branching out in four directions.

### Example 2

As illustrated in FIG. 5B, straight partial storage channels 31 are disposed to successively branch out in a fan-like fashion at branched portions 33, each of which is in communication with the partial storage channels 31 branching out in three directions. The inner wall surfaces of the partial storage channels 31 in a conduction path join together via the inner wall surfaces of the branched portion 33.

### Example 3

As illustrated in FIG. 5C, two straight partial storage channels 31 are successively joined to a semicircular inflecting portion 35 at bending inflecting portions 35 to form a continuous, semicircular concentric spiral.

### Example 4

As illustrated in FIG. 5D, straight partial storage channels 31 and semicircular inflecting portions 35 are joined at large numbers of branched portions 33 to branch out in four directions at the branched portions 33 in a concentric circle.

### Example 5

As illustrated in FIG. 5E, arc-shaped inflecting portions 35, and straight partial storage channels 31 radially extending out from the center of the arc are joined at branched portions 33, so that each branched portion 33 is in communication with the arc-shaped inflecting portions 35 and the straight partial storage channels 31 branching out in three or four directions in a half circle with radiating lines.

### Example 6

As illustrated in FIG. 5F, large numbers of straight partial storage channels 31 are joined at a right angle to form a lattice so that the straight partial storage channels 31 branch out in four directions at branched portions 33 in communication therewith.

### Example 7

As illustrated in FIG. 5G, large numbers of straight partial storage channels 31 are crossed so that the straight partial storage channels 31 join or branch out at branched portions 33 and bent portions 34. Each branched portion 33 is in communication with the straight partial storage channels 31 branching out in four directions. Each bent portion 34 is in communication with the straight partial storage channels 31 branching out in two directions.

The observation of the pseudo blood dropped on each test piece of Examples 1 to 7 revealed that the storage channels 15 of these examples are all capable of transferring and storing the pseudo blood.

### Comparative Example 1

The state of the pseudo blood being transferred and stored was observed as in Examples 1 to 7, except that a circular recess (diameter, 500 μm; depth, 50 μm) was provided at the end of each collection channel 17 instead of the storage channel 15. The maximum cross sectional area of the circular recess was greater than that of the collection channels 17 by a factor of about 42.

The transfer of the pseudo blood stopped at the end of the collection channels 17, and the pseudo blood was not stored in the circular recess.

### Comparative Example 2

The state of the pseudo blood being transferred and stored was observed as in Example 1, except that the width of the storage channels 15 orthogonal to the axis line was 20 μm over the entire length, that the depth of the partial storage channels 31 between the collection channels 17 and the branched portions 33 was 40 μm, that the depth of the partial storage channels in communication with one another via the branched portions 33 was 50 μm, and that these partial storage channels were discontinuous at the branched portions 33.

The transfer of the pseudo blood stopped at the branched portions 33, and the pseudo blood was not stored in the partial storage channel 31.

### NUMERALS

10 Apparatus for containing sampled liquid
11 Substrate
13 Puncture portion
15 Storage channel
17 Collection channel
25 jagged surface portion
31 Partial storage channel
31a Bottom surface
33 Branched portion
33a Bottom surface
34 Bent portion
35 Curved portion

What is claimed is:

1. An apparatus for containing sampled liquid, comprising:
a substrate; and
a puncture portion, integral with the substrate, and that projects out of the substrate for insertion into an organism;
the puncture portion including one or more collection channels, and the substrate including a storage channel in communication with the one or more collection channels,
the one or more collection channels collecting a compositional bodily fluid, and the storage channel storing the compositional bodily fluid transferred thereto by capillary action,
wherein the storage channel includes an inflecting portion and/or a branched portion shaped to enable capillary action transfer of the compositional bodily fluid, and wherein at least an innermost portion of the storage channel farthest from the one or more collection channels is open to atmosphere.

2. An apparatus for containing sampled liquid according to claim 1, wherein at least a part of an inner wall surface of the storage channel is continuous in the inflecting portion and/or the branched portion without substantially any step.

3. An apparatus for containing sampled liquid according to claim 1, wherein at least a part of an inner wall surface of the storage channel is continuous over substantially an entire length of the storage channel without substantially any step.

4. An apparatus for containing sampled liquid according to claim 1, wherein the storage channel is a form of a groove open to atmosphere over its entire length.

5. An apparatus for containing sampled liquid according to claim 4, wherein the storage channel includes a portion into which the compositional bodily fluid is introduced in a plurality of directions.

6. An apparatus for containing sampled liquid according to claim 1, wherein the storage channel includes a plurality of partial storage channels in communication with one another via the branched portion, and wherein the partial storage channels are formed so that a maximum value-to-minimum value ratio of a cross sectional area of the partial storage channels orthogonal to an axis line of the storage channel is 2.5 or less at ends adjacent to the branched portion in communication with the partial storage channels.

7. An apparatus for containing sampled liquid according to claim 1, wherein a maximum value-to-minimum value ratio of a cross sectional area of the storage channel orthogonal to its axis line is 2.5 or less except at the branched portion.

8. An apparatus for containing sampled liquid according to claim 1, wherein a cross sectional shape of the storage channel orthogonal to its axis line is uniform except at the inflecting portion and the branched portion.

9. An apparatus for containing sampled liquid according to claim 1, wherein a cross sectional shape of the storage channel orthogonal to its axis line is substantially quadrangular.

10. An apparatus for containing sampled liquid according to claim 9, wherein at least one of inner wall surfaces of the storage channel is a flat, continuous surface in a two-dimensional plane over an entire length of the storage channel.

11. An apparatus for containing sampled liquid according to claim 1, wherein the apparatus has a plurality of the collection channels.

12. An apparatus for containing sampled liquid according to claim 1, wherein the puncture portion includes an jagged surface on at least a portion of its lateral periphery, and wherein the one or more collection channels is a form of a slit or a groove with a continuous opening on the jagged surface.
